(19) United States
(12) Patent Application Publication
(10) Pub. No.: US 2004/0229939 A1
(43) Pub. Date: Nov. 18, 2004

(54) Tetrahydrocannabinol compositions and methods of manufacture and use thereof

(76) Inventors: Dipak K. Chowdhury, Lexington, KY (US); B. Ram Murty, Lexington, KY (US)

Correspondence Address: George David McClure, Jr., P. P. Box 21902, Lexington, KY, 40522, US

(21) Appl. No.: 10/780,121

(22) Filed: Feb. 17, 2004

Related U.S. Application Data
(63) Continuation-in-part of application No. 10/724,337, filed on Nov. 28, 2003.
(60) Provisional application No. 60/447,413, filed on Feb. 14, 2003.
(60) Provisional application No. 60/447,414, filed on Feb. 14, 2003.

(51) Int. Cl. A61K 31/353

(52) U.S. Cl. 514/454

(57) ABSTRACT

Disclosed is a sublingual pharmaceutical formulation containing tetrahydrocannabinol and certain excipients. Also disclosed is how to make and use the formulation.

### CROSS-REFERENCE TO RELATED APPLICATIONS

This application is a continuation-in-part of application Ser. No. 10/724,337, filed Nov. 28, 2003.

### PRIORITY

Priority is claimed on the basis of provisional application Nos. 60/447,413 and 60/447,414, filed Feb. 14, 2003, which are fully incorporated herein by reference in their entirety.

### STATEMENT REGARDING FEDERAL SPONSORSHIP

Not applicable

### FIELD OF THE INVENTION

The invention relates to tetrahydrocannabinol compositions and methods of manufacture and use thereof.

### BACKGROUND OF THE INVENTION

Hundreds of medically useful compounds are discovered each year, but clinical use of these drugs is possible only if a drug delivery vehicle is developed to transport them to their therapeutic target in the human body. This problem is particularly critical for water-insoluble or poorly soluble drugs. For such hydrophobic compounds, direct injection may be highly dangerous and can result in hemolysis, phlebitis, hypersensitivity, organ failure, or death. Tetrahydrocannabinol (“THC”) is one such compound.

While THC, especially Delta 9-tetrahydrocannabiriol, is useful in treating, lessening, or ameliorating emesis, anorexia, or chronic or AIDS-related wasting syndrome in a subject in which it is desired to treat, to lessen, or to ameliorate emesis, anorexia, or chronic or AIDS-related wasting syndrome, THC is so poorly soluble in water that it is difficult to prepare therapeutically useful aqueous formulations of THC at THC concentrations such as 2 micrograms per milliliter. It is an object of the invention to provide a therapeutically useful aqueous formulation of THC.

THC is effective in treating pain, nausea and vomiting associated with chemotherapy and severe weight loss associated with AIDS. It has been recommended that THC be administered to patients who have not responded to other therapies for these conditions.

There is a dearth of THC-based pharmaceuticals on the market. One marketed THC-based pharmaceutical is available in capsule dosage form for oral administration and was approved by the US Food and Drug Administration for indications including emesis associated with chemotherapy and severe weight loss associated with AIDS. However, oral therapy frequently results in a poor or partial response. This may be due to the limited aqueous solubility of THC and its extensive first-pass metabolism following oral administration. Thus, absolute bioavailability of Delta 9-THC is low. In addition, fasting or food deprivation can decrease the rate of absorption of THC from the currently marketed sesame oil capsules. There is also large inter-subject variability in absorption. For this reason it may be important to titrate the THC dose on an individual basis, since the drug has biphasic activity and a narrow therapeutic index.

THC has been utilized throughout the world for centuries. THC appears to be efficacious for the amelioration of nausea due to chemotherapy and for the management of chronic pain. THC can even be utilized to reduce the devastating inflammatory process caused by acute injury to the brain or spinal cord.

Physiologically active constituents of marijuana include the two tetrahydrcannabinols, Delta 9-tetrahydrocannabinol and Delta 8-tetrahydrocannabinol. Water-soluble derivatives have been obtained by esterification of the phenolic group.

The pharmacokinetics of THC varies with the route of administration. When smoked, Delta 9-THC is rapidly absorbed by the blood in the lungs. Oral absorption of THC is less rapid than from the lungs. The disappearance of Delta 9-THC from the blood following intravenous (IV) administration is biphasic. High blood levels fall rapidly for the first 30 minutes as the Delta 9-THC distributes to tissues with high blood flow. After the initial high distribution, the blood level falls much more slowly with a half-life of 19 hours or more. After an IV injection of a single dose of Delta 9-THC, approximately 25-30 percent of the compound and its metabolites remain in the body for one week. In addition, blood levels of Delta 9-THC are higher and last longer when given in an oily solution than in an ethyl alcohol solution. This suggests that cannabis taken with food mixtures containing fat is better absorbed.

An important difference between smoking and ingestion as means of THC administration is that when cannabinoids are absorbed from the gut, the blood containing them first goes directly through the liver. The liver rapidly clears the Delta 9-THC from the blood and enzymatically changes much of the Delta 9-THC to other metabolites before much of the Delta 9-THC can reach the brain. A large proportion is metabolized to 11-hydroxy delta 9-THC. When taken orally, two to three times more Delta 9-THC is required to obtain equivalent acute psychological and physiological effects, as compared with THC administered by smoking.

Apart from this, patients who suffer from severe pain after surgery are given painkillers, such as morphine, which are known to induce vomiting. To reduce vomiting, it is essential to administer an antiemetic agent that can act rapidly. In an attempt to overcome such problems, transdermal patches have been proposed. For example, U.S. Pat. No. 6,113,940 discloses a patch-like device by means of which cannabinoids are delivered transdermally. It can be seen, however, that transdermal approaches have certain limitations, such as variation in the amount of THC released. Since THC has a narrow therapeutic index, it may reach toxic levels if there is too much variation of release.

It is therefore an object of the invention to provide a composition useful for safe, reliable and effective delivery of THC.

References concerning the foregoing background include the following:

Physician's Desk Reference. 50th ed, Medical economics data, Oradell, N.J., 611 (1966).

Cohen, S. In Marijuana Research Findings, Petersen, R. C. (Ed), NIDA RES. Monogr. 31, DHHS, 1980.

Scadler, B. M., Wall, M. E., Perez-Reyes. In the Cannabinoids: Chemical, Pharmacologic and therapeutics aspects, Agurell, S., Dewey, W. L., Willette, R. E. (Eds). Academic New York, pp227, 1984 4. Joy, J. E., Watson, S. J. And Benson, J. A. Marijuana and Medicine: Assessing the Science Base. National Academy Press, Washington. D.C. 1999.

Pryor, G. T., and Mitoma, C. Influence of fasting on the absorption and effects of Delta 9-tetrahydrocannabinol after oral administration in sesame oil. Pharmacol. Biochem. Behav. 6: 331-441(1997).

Ohlsson, A., Lindergren J. E., Wahlen, A., Plasma delta 9-tetrahydrocannabinol concentrations and clinical effects after oral and intravenous administration and smoking. Clin. Pharmacol. Ther. 28: 409-416(1980).

The Merck Index, Merck and co. Inc. Rahway, New Jersy (1989).

Hunt, A. and Jones, R. T. Tolerance and disposition of tetrahydrocannabinol in man Pharmacol. Exp. Ther. 215, 35-44 (1980).

Perez-Reyes, M., Lipton M. A., Timmons M. C. Pharmacology of orally administered Delta 9-THC. Clin. Pharmacol. Ther. 14, 48-55, 1973.

### DESCRIPTION OF THE INVENTION

Accordingly, the invention provides a pharmaceutical composition comprising tetrahydrocannabinol, ethanol, and a pharmaceutically acceptable excipient.

In an initial embodiment, the invention provides a pharmaceutical composition comprising THC, ethanol, microcrystalline cellulose, sodium starch glycolate, magnesium stearate, fumed silica, and any of the group consisting of mannitol, sucrose, lactose, sorbitol, lactitol, and xylitol.

In a second embodiment, the invention provides a pharmaceutical composition comprising (a) THC, ethanol, sodium bicarbonate, sodium carbonate, magnesium stearate, and fumed silica; (b) any of the group consisting of citric acid and tartaric acid; and (c) any of the group consisting of mannitol, sucrose, lactose, sorbitol, lactitol, and xylitol.

In a third embodiment, the invention provides a pharmaceutical composition comprising THC, ethanol, magnesium stearate, and fumed silica, and further comprising, by mass, from about 0.01% to about 0.05% sodium starch glycolate, from about 0.1% to about 0.5% microcrystalline cellulose, and from about 0.1% to about 0.5% any of the group consisting of mannitol, sucrose, lactose, sorbitol, lactitol, and xylitol.

In a fourth embodiment, the invention provides a pharmaceutical composition comprising (a) THC, ethanol, sodium carbonate, magnesium stearate, and fumed silica; (b) any of the group consisting of mannitol, sucrose, lactose, sorbitol, lactitol, and xylitol; (c) by mass, from about 30% to about 40% sodium bicarbonate; and (d) by mass, from about 15% to about 25% any of the group consisting of citric acid and tartaric acid, wherein the mass ratio of sodium carbonate to sodium bicarbonate is from about 1:3 to about 1:4.

In a fifth embodiment, the invention provides a pharmaceutical composition according to the any of the foregoing embodiments and further comprising a water-soluble surfactant.

In a sixth embodiment, the invention provides a pharmaceutical composition according to the fifth embodiment, wherein the water-soluble surfactant is a member of the group consisting of sodium lauryl sulfate, polysorbate 80 (Tween 80), polyoxyethylene-polyoxypropylene block copolymer (Poloxamer 188, 407, 237) and beta cyclodextrins (hydroxypropyl beta cyclodextrin).

In a seventh embodiment, the invention provides a pharmaceutical composition according to the any of the foregoing embodiments and further comprising either water soluble or water insoluble antioxidant.

In an eighth embodiment, the invention provides a pharmaceutical composition according to any of the foregoing embodiments and further comprising a member of the group consisting of: a sweetening agent, a coloring agent, and a flavoring agent.

In a ninth embodiment, the invention provides a method of treating, preventing, ameliorating, lessening or mitigating nausea or emesis comprising administering to a subject in need of said treating, preventing, ameliorating, lessening or mitigating, a therapeutically effective amount of a pharmaceutical composition according to any of the foregoing embodiments.

In a tenth embodiment, the invention provides a method of treating, preventing, ameliorating, lessening or mitigating nausea or emesis according to the eighth embodiment, wherein a substantially therapeutically effective amount of the THC of the pharmaceutical composition (a) is absorbed by the subject in a period not greater than about two minutes or (b) substantially disintegrates or dissolves in the oral cavity of the subject in a period not greater than about one minute.

For example, to prepare a batch of 100 tablets, 360 mg of THC and 0.2 g butyl hydroxytoluene (“BHT”) were dissolved in 5 mL ethanol and slowly added to 2.5 g mannitol to form a granular mixture. Then 3.5 g sodium bicarbonate and 1.0 g sodium carbonate were mixed to form a first mixture. Then the granular mixture was added to the first mixture with trituration and placed in a tray dryer to form a powder mixture. Then 0.1 g sodium lauryl sulfate and 0.3 g sodium saccharin were mixed and added to the drying mixture to form a penultimate mixture. Then 0.1 g magnesium stearate was added to the penultimate mixture and mixed with the penultimate mixture to form a final mixture. The final mixture was then compressed into tablets. The tablets were found to possess a release profile consistent with their usefulness in a method according to the tenth embodiment.

For example, to prepare a batch of 100 tablets, 255 mg of THC and 0.2 g butyl hydroxytoluene were dissolved in 1.2 mL ethanol and slowly added to 2.5 g mannitol and kept in a tray dryer at 35 C for 10 min, thereby forming an initial granular mixture. Then 2.0 g citric acid monohydrate and 3.5 g sodium bicarbonate were triturated and passed through a #40 sieve to form a first sieved mixture. Then 1.0 g sodium carbonate were passed through the sieve and mixed with the first sieved mixture to form a second sieved mixture. Then the initial granular mixture was passed through the sieve and added to the second sieved mixture to form a third sieved mixture. Then 0.1 g sodium lauryl sulfate and 0.3 g sodium saccharin were passed through the sieve and added to the third sieved mixture to form a fourth sieved mixture. Then 0.1 g magnesium stearate was added to the fourth sieved mixture and mixed with the fourth sieved mixture to form an ultimate mixture. The ultimate mixture was then compressed into tablets. The tablets were found to possess a release profile consistent with their usefulness in a method according to the tenth embodiment.

Each of the foregoing embodiments is merely exemplary and is not intended to limit the scope of the invention, which encompasses all equivalents of what is described herein and set forth in the following claims.

What is claimed is:

1. A pharmaceutical formulation for sublingual delivery of tetrahydrocannabinol and suitable for tableting, the formulation comprising tetrahydrocannabinol, ethanol, a buffer, an antioxidant, a water-soluble excipient, a detergent, a sweetener, and a glidant.

2. A formulation according to claim 1, wherein the buffer comprises a member of the group consisting of citrate buffers and carbonate buffers.

3. A formulation according to claim 1, wherein the antioxidant comprises BHT.

4. A formulation according to claim 1, wherein the water-soluble excipient comprises mannitol.

5. A formulation according to claim 1, wherein the detergent comprises sodium lauryl sulfate.

6. A formulation according to claim 1, wherein the sweetener comprises sodium saccharin.

7. A formulation according to claim 1, wherein the glidant comprises magnesium stearate.

8. A method of treating, lessening, or ameliorating emesis, anorexia, or chronic or AIDS-related wasting syndrome in a subject in which it is desired to treat, to lessen, or to ameliorate emesis, anorexia, or chronic or AIDS-related wasting syndrome, said method comprising administering to the subject a therapeutically effective amount of a formulation according to claim 1.
